(19) United States
(12) Patent Application Publication
(10) Pub. No.: US 2008/0146915 A1
(43) Pub. Date: Jun. 19, 2008

(54) SYSTEMS AND METHODS FOR VISUALIZING A CANNULA TRAJECTORY

(76) Inventor: Gerald McMorrow, Redmond, WA (US)

Correspondence Address: BLACK LOWE & GRAHAM, PLLC, 701 FIFTH AVENUE, SUITE 4800, SEATTLE, WA, 98104, US

(21) Appl. No.: 11/874,824

(22) Filed: Oct. 18, 2007

Related U.S. Application Data
(60) Provisional application No. 60/862,182, filed on Oct. 19, 2006.

(51) Int. Cl. A61B 8/00

(52) U.S. Cl. 600/424; 600/437

(57) ABSTRACT

A system and method for visualizing a cannula trajectory. An embodiment of the present invention generally includes an ultrasound probe attached to a first camera and/or a second camera and a processing and display generating system that may be in signal communication with the ultrasound probe, the first camera, and/or the second camera. A user of the system scans tissue containing a target vein using the ultrasound probe and a cross-sectional image of the target vein may be displayed. The first camera records a first image of a cannula in a first direction and the second camera records a second image of the cannula in a second direction orthogonal to the first direction. The first and/or the second images may be processed by the processing and display generating system along with the relative positions of the ultrasound probe, the first camera, and/or the second camera to determine the trajectory of the cannula. A representation of the determined trajectory of the cannula may be then displayed on the ultrasound image.

### RELATED APPLICATIONS

This application claims priority to and incorporates by reference U.S. Provisional Patent Application Ser. No. 60/862,182 filed Oct. 19, 2006.

### FIELD OF THE INVENTION

The invention relates to visualization methods and systems, and more specifically to systems and methods for visualizing the trajectory of a cannula or needle being inserted in a biologic subject.

### BACKGROUND OF THE INVENTION

Unsuccessful insertion and/or removal of a cannula, a needle, or other similar devices into vascular tissue may cause vascular wall damage that may lead to serious complications or even death. Image-guided placement of a cannula or needle into the vascular tissue reduces the risk of injury and increases the confidence of healthcare providers in using the foregoing devices. Current image guided placement methods generally use a guidance system for holding specific cannula or needle sizes. The motion and force required to disengage the cannula from the guidance system may, however, contribute to a vessel wall injury, which may result in extravasation. Complications arising from extravasation resulting in morbidity are well documented. Therefore, there is a need for image guided placement of a cannula or needle into vascular tissue while still allowing a health care practitioner to use standard “free” insertion procedures that do not require a guidance system to hold the cannula or needle.

### BRIEF DESCRIPTION OF THE DRAWINGS

FIGS. 1 and 2 are diagrams showing one embodiment of the present invention;

FIG. 3 is a diagram showing additional detail for a needle shaft to be used with one embodiment of the invention;

FIGS. 4A and 4B are diagrams showing close-up views of surface features of the needle shaft shown in FIG. 3;

FIG. 5 is a diagram showing imaging components for use with the needle shaft shown in FIG. 3;

FIG. 6 is a diagram showing a representation of an image produced by the imaging components shown in FIG. 5;

FIG. 7 is a system diagram of an embodiment of the present invention;

FIG. 8 is a system diagram of an example embodiment showing additional detail for one of the components shown in FIG. 2;

FIGS. 9-10 are flowcharts of a method of displaying the trajectory of a cannula in accordance with an embodiment of the present invention; and

FIG. 11 schematically depicts an alternative embodiment of a needle having a distribution of reflectors located near a bevel of the needle.

### DETAILED DESCRIPTION OF THE PREFERRED EMBODIMENT

An example embodiment includes a system and method using single or multiple cameras for tracking and displaying the movement of a needle or cannula before and/or during insertion into a blood vessel or other sub-dermal structure and subsequent movements therein. A needle or a cannula-fitted needle may be detachably mounted to an ultrasound transceiver in signal communication with a computer system and display configured to generate ultrasound-acquired images and process images received from the single or multiple cameras. Along the external surfaces of the needle or cannula may be fitted optical reflectors that may be discernable in the camera images. The ultrasound transceiver may be secured against a subject's dermal area adjacent to a sub-dermal region of interest (ROI). Optical signals may be reflected towards the single or multiple cameras by the needle or cannula embedded reflectors and conveyed to the computer system and display. The trajectories of the needle or cannula movements may be determined by data analysis of the reflector signals detected by the cameras. The trajectories of needle or cannula having one or more reflectors may be overlaid onto the ultrasound images to provide alignment coordinates for insertion of the needle or cannula fitted needle into the ROI along a determined trajectory.

An example embodiment of the present invention generally includes an ultrasound probe attached to a first camera and a second camera. The example embodiment also generally includes a processing and display generating system that may be in signal communication with the ultrasound probe, the first camera, and/or the second camera. Typically, a user of the system scans tissue containing a target vein using the ultrasound probe and a cross-sectional image of the target vein may be displayed. The first camera captures and/or records a first image of a medical object to be inserted, such as a cannula for example, in a first direction and the second camera captures and/or records a second image of the cannula in a second direction orthogonal to the first direction. The first and/or the second images may be processed by the processing and display generating system along with the relative positions of the ultrasound probe, the first camera, and/or the second camera to determine the trajectory of the cannula. A representation of the determined trajectory of the cannula may be then displayed on the ultrasound image.

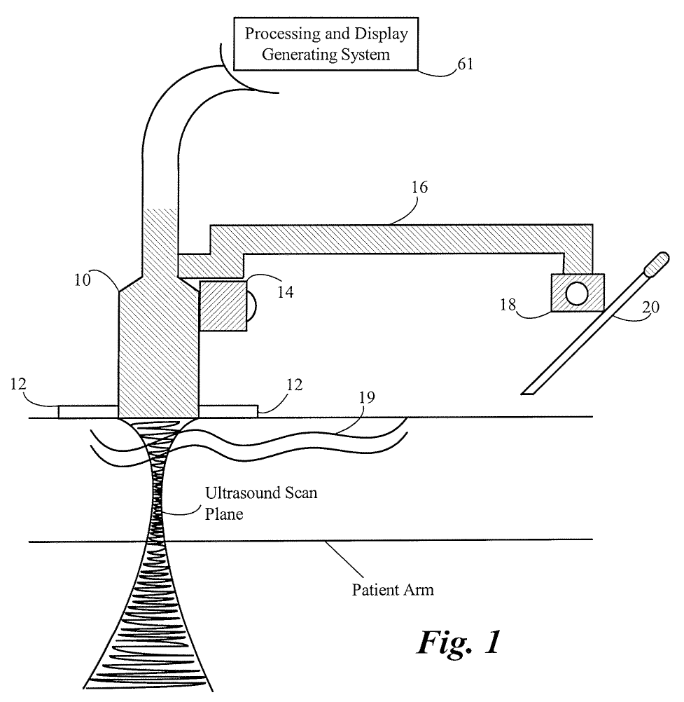

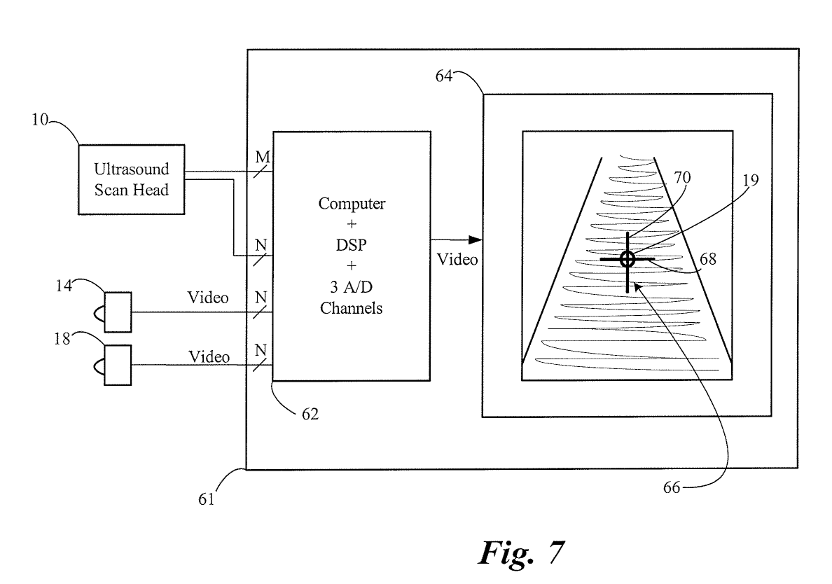

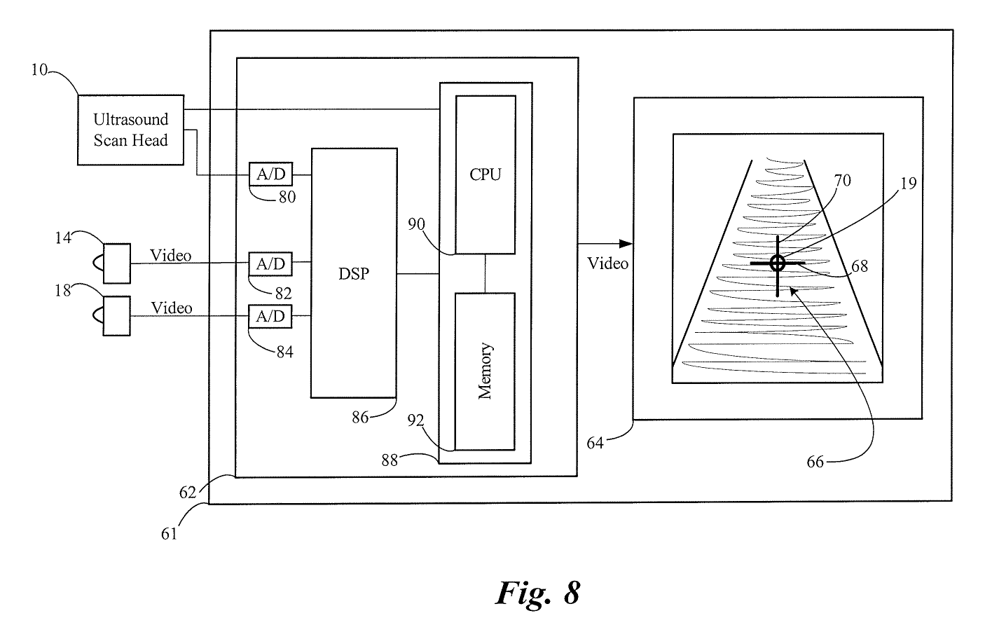

FIG. 1 is a diagram illustrating a side view of one embodiment of the present invention. A two-dimensional (2D) ultrasound probe 10 may be attached to a first camera 14 that takes images in a first direction. The ultrasound probe 10 may be also attached to a second camera 18 via a member 16. In other embodiments, the member 16 may link the first camera 14 to the second camera 18 or the member 16 may be absent, with the second camera 18 being directly attached to a specially configured ultrasound probe. The second camera 18 may be oriented such that the second camera 18 takes images in a second direction that may be orthogonal to the first direction of the images taken by the first camera 14. The placement of the cameras 14, 18 may be such that they can both take images of a cannula 20 when the cannula 20 may be placed before the cameras 14, 18. A needle may also be used in place of a cannula. The cameras 14, 18 and the ultrasound probe 10 may be geometrically interlocked such that the cannula 20 trajectory can be related to an ultrasound image. In FIG. 1, the second camera 18 may be behind the cannula 20 when looking into the plane of the page. In an embodiment, the cameras 14, 18 take images at a rapid frame rate of approximately 30 frames per second. The ultrasound probe 10 and/or the cameras 14, 18 may be in signal communication with a processing and display generating system 61 described in FIGS. 7 and 8 below.

In typical operation, a user first employs the ultrasound probe 10 and the processing and display generating system 61 to generate a cross-sectional image of a patient's arm tissue containing a vein to be cannulated (“target vein”) 19. This could be done by one of the methods disclosed in the patents, patent publications and/or patent applications which are herein incorporated by reference, such as, for example, U.S. patent application Ser. No. 11/460,182 filed Jul. 26, 2006. The user then identifies the target vein 19 in the image using methods such as simple compression which differentiates between arteries and/or veins by using the fact that veins collapse easily while arteries do not. After the user has identified the target vein 19, the ultrasound probe 10 may be affixed to the patient's arm over the previously identified target vein 19 using a magnetic tape material 12, for example. The ultrasound probe 10 and the processing and display generating system 61 continue to generate a 2D cross-sectional image of the tissue containing the target vein 19. Images from the cameras 14, 18 may be provided to the processing and display generating system 61 as the cannula 20 may be approaching and/or entering the arm of the patient.

The processing and display generating system 61 locates the cannula 20 in the images provided by the cameras 14, 18 and determines the projected location at which the cannula 20 will penetrate the cross-sectional ultrasound image being displayed. The trajectory of the cannula 20 may be determined in some embodiments by using image processing to identify bright spots corresponding to micro reflectors previously machined into the shaft of the cannula 20 or a needle used alone or in combination with the cannula 20. Image processing uses the bright spots to determine the angles of the cannula 20 relative to the cameras 14, 18 and then generates a projected trajectory by using the determined angles and/or the known positions of the cameras 14, 18 in relation to the ultrasound probe 10. In other embodiments, determination of the cannula 20 trajectory may be performed using edge-detection algorithms in combination with the known positions of the cameras 14, 18 in relation to the ultrasound probe 10, for example.

The projected location may be indicated on the displayed image as a computer-generated cross-hair 66 (shown in FIG. 7), the intersection of which may be where the cannula 20 is projected to penetrate the image. In other embodiments, the projected location may be depicted using a representation other than a cross-hair. When the cannula 20 does penetrate the cross-sectional plane of the scan produced by the ultrasound probe 10, the ultrasound image confirms that the cannula 20 penetrated at the location of the cross-hair 66. This gives the user a real-time ultrasound image of the target vein 19 with an overlaid real-time computer-generated image of the position in the ultrasound image that the cannula 20 will penetrate. This allows the user to adjust the location and/or angle of the cannula 20 before and/or during insertion to increase the likelihood they will penetrate the target vein 19. In other embodiments, the ultrasound image and/or the computer-generated cross-hair may be displayed in near real-time. In an example embodiment, this allows a user to employ normal “free” insertion procedures while having the added knowledge of knowing where the cannula 20 trajectory will lead.

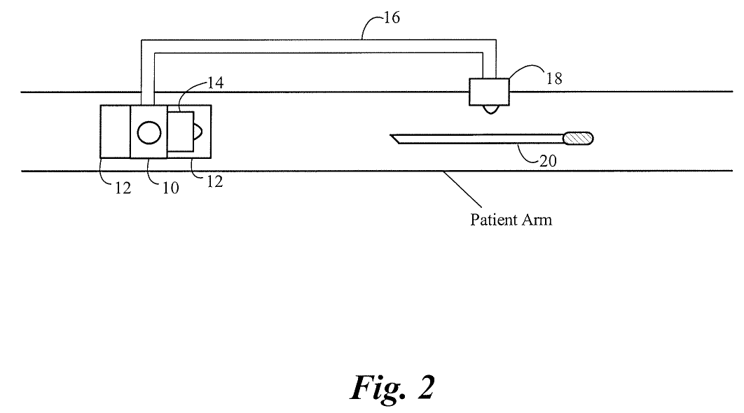

FIG. 2 is a diagram illustrating a top view of the embodiment shown in FIG. 1. It is more easily seen from this view that the second camera 18 may be positioned behind the cannula 20. The positioning of the cameras 14, 18 relative to the cannula 20 allows the cameras 14, 18 to capture images of the cannula 20 from two different directions, thus making it easier to determine the trajectory of the cannula 20.

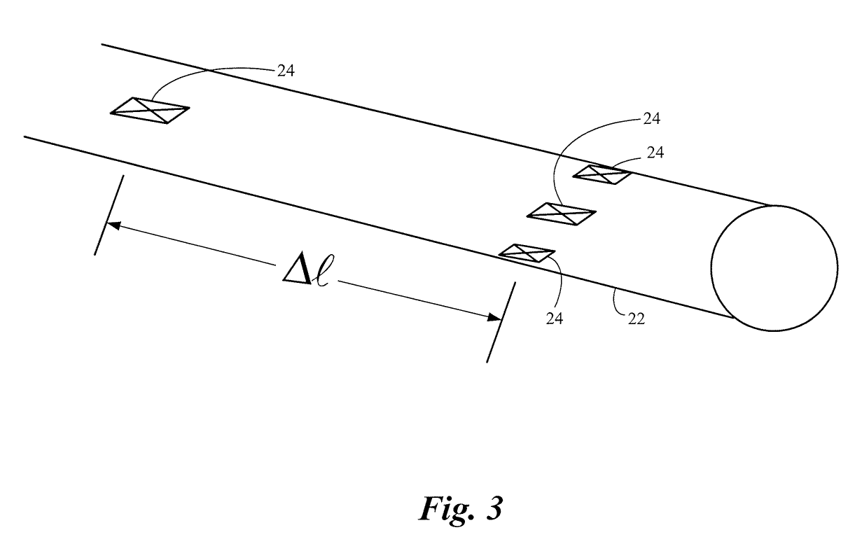

FIG. 3 is diagram showing additional detail for a needle shaft 22 to be used with one embodiment of the invention. The needle shaft 22 includes a plurality of micro corner reflectors 24. The micro corner reflectors 24 may be cut into, or otherwise affixed to or embedded in, the needle shaft 22 at defined intervals Δl in symmetrical patterns about the circumference of the needle shaft 22. The micro corner reflectors 24 could be cut with a laser, for example.

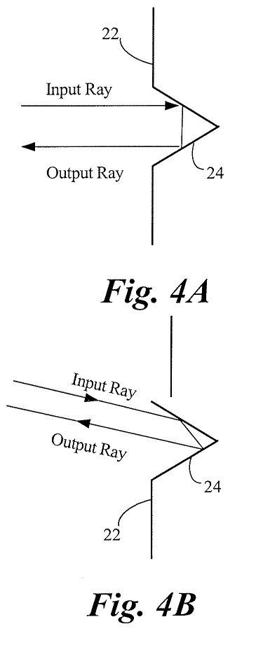

FIGS. 4A and 4B are diagrams showing close-up views of surface features of the needle shaft 22 shown in FIG. 3. FIG. 4A shows a first input ray with a first incident angle of approximately 90° striking one of the micro corner reflectors 24 on the needle shaft 22. A first output ray is shown exiting the micro corner reflector 24 in a direction toward the source of the first input ray. FIG. 4B shows a second input ray with a second incident angle other than 90° striking a micro corner reflector 25 on the needle shaft 22. A second output ray is shown exiting the micro corner reflector 25 in a direction toward the source of the second input ray. FIGS. 4A and 4B illustrate that the micro corner reflectors 24, 25 are useful because they tend to reflect an output ray in the direction from which an input ray originated.

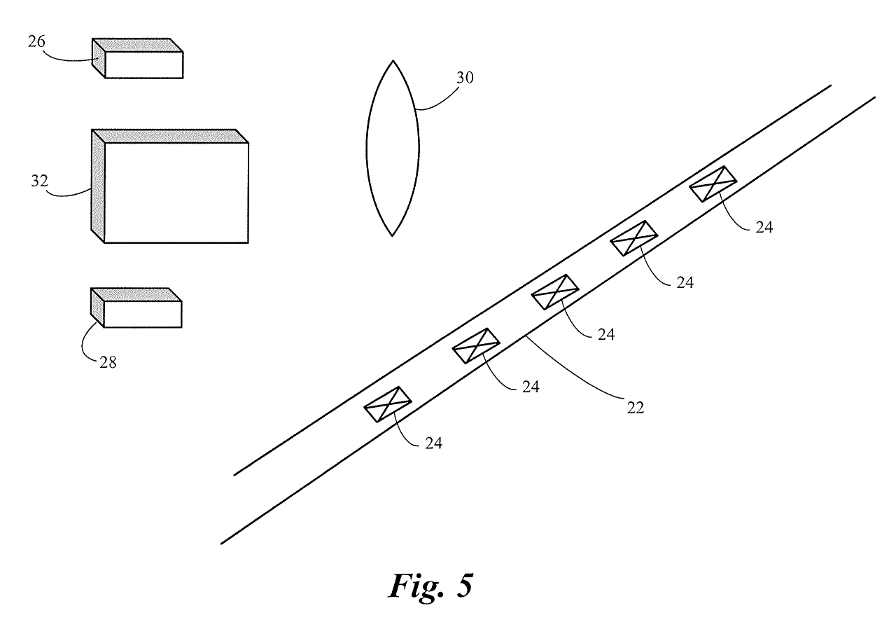

FIG. 5 is a diagram showing imaging components for use with the needle shaft 22 shown in FIG. 3 in accordance with an example embodiment of the invention. The imaging components are shown to include a first light source 26, a second light source 28, a lens 30, and a sensor chip 32. The first and/or second light sources 26, 28 may be light emitting diodes (LEDs), for example. In an example embodiment, the light sources 26, 28 are infra-red LEDs. Use of an infra-red source is advantageous because it is not visible to the human eye, but when an image of the needle shaft 22 is recorded, the image can show strong bright dots where the micro corner reflectors 24 may be located because silicon sensor chips are sensitive to infra-red light and the micro corner reflectors 24 tend to reflect output rays in the direction from which input rays originate, as discussed with reference to FIGS. 4A and 4B. In alternative embodiments, a single light source may be used. Although not shown, the sensor chip 32 may be encased in a housing behind the lens 30 and the sensor chip 32 and light sources 26, 28 may be in electrical communication with the processing and display generating system 61 shown in FIG. 7 below. The sensor chip 32 and/or the lens 30 form a part of the first and second cameras 14, 18 in some embodiments. In an example embodiment, the light sources 26, 28 may be pulsed on at the time the sensor chip 32 captures an image. In other embodiments, the light sources 26, 28 may be left on during video image capture.

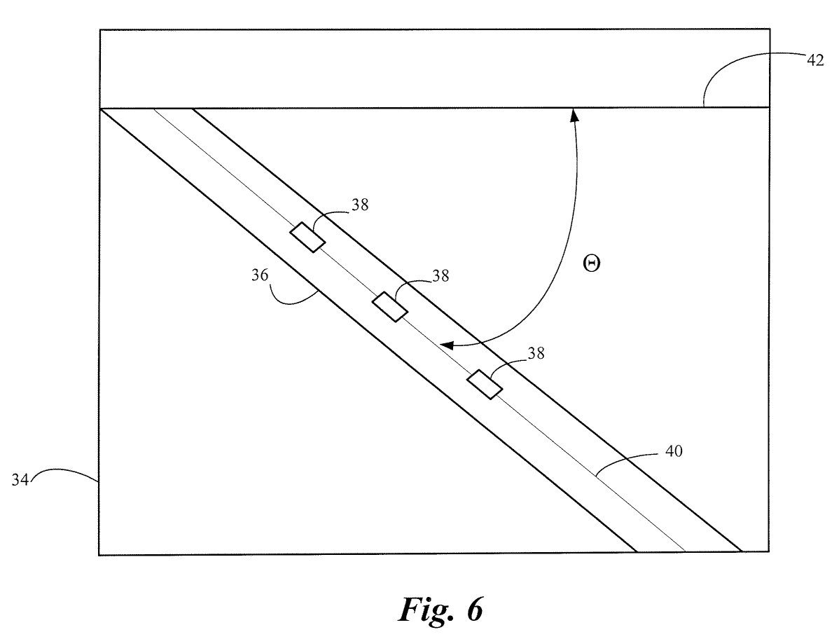

FIG. 6 is a diagram showing a representation of an image 34 produced by the imaging components shown in FIG. 5. The image 34 may include a needle shaft image 36 that corresponds to a portion of the needle shaft 22 shown in FIG. 5. The image 34 also may include a series of bright dots 38 running along the center of the needle shaft image 36 that correspond to the micro corner reflectors 24 shown in FIG. 5. A center line 40 is shown in FIG. 6 that runs through the center of the bright dots 38. The center line 40 may not appear in the actual image generated by the imaging components, but is shown in the diagram to illustrate how an angle theta (θ) could be obtained by image processing to recognize the bright dots 38 and determine a line through them. The angle theta represents the degree to which the needle shaft 22 may be inclined with respect to a reference line 42 that may be related to the fixed position of the sensor chip 32.

FIG. 7 is a system diagram of an embodiment of the present invention and shows additional detail for the processing and display generating system 61 in accordance with an example embodiment of the invention. The ultrasound probe 10 is shown connected to the processing and display generating system via M control lines and N data lines. The M and N variables are for convenience and appear simply to indicate that the connections may be composed of one or more transmission paths. The control lines allow the processing and display generating system 61 to direct the ultrasound probe 10 to properly perform an ultrasound scan and the data lines allow responses from the ultrasound scan to be transmitted to the processing and display generating system 61. The first and second cameras 14, 18 are also each shown to be connected to the processing and display generating system 61 via N lines. Although the same variable N is used, it is simply indicating that one or more lines may be present, not that each device with a label of N lines has the same number of lines.

The processing and display generating system 61 may be composed of a display 64 and a block 62 containing a computer, a digital signal processor (DSP), and analog to digital (A/D) converters. As discussed for FIG. 1, the display 64 can display a cross-sectional ultrasound image. The computer-generated cross hair 66 is shown over a representation of a cross-sectional view of the target vein 19 in FIG. 7. The cross hair 66 consists of an x-crosshair 68 and a z-crosshair 70. The DSP and the computer in the block 62 use images from the first camera 14 to determine the plane in which the cannula 20 will penetrate the ultrasound image and then write the z-crosshair 70 on the ultrasound image provided to the display 64. Similarly, the DSP and the computer in the block 62 use images from the second camera 18, which may be orthogonal to the images provided by the first camera 14 as discussed for FIG. 1, to write the x-crosshair 68 on the ultrasound image. In other embodiments, the DSP and the computer in the block 62 may use images from both the first camera 14 and the second camera 18 to write each of the x-crosshair 68 and the z-crosshair 70 on the ultrasound image. In still other examples, images from the cameras 14, 18 may be used separately or in combination to write the crosshairs 68, 70 or other representations of where the cannula 20 is projected to penetrate the ultrasound image.

FIG. 8 is a system diagram of an example embodiment showing additional detail for the block 62 shown in FIG. 2. The block 62 includes a first A/D converter 80, a second A/D converter 82, and a third A/D converter 84. The first A/D converter 80 receives signals from the ultrasound probe 10 and converts them to digital information that may be provided to a DSP 86. The second and third A/D converters 82, 84 receive signals from the first and second cameras 14, 18 respectively and convert the signals to digital information that may be provided to the DSP 86. In alternative embodiments, some or all of the A/D converters are not present. For example, video from the cameras 14, 18 may be provided to the DSP 86 directly in digital form rather than being created in analog form before passing through A/D converters 82, 84. The DSP 86 may be in data communication with a computer 88 that includes a central processing unit (CPU) 90 in data communication with a memory component 92. The computer 88 may be in signal communication with the ultrasound probe 10 and may be able to control the ultrasound probe 10 using this connection. The computer 88 may be also connected to the display 64 and may produce a video signal used to drive the display 64. In still other examples, other hardware components may be used. A field programmable gate array (FPGA) may be used in place of the DSP, for example. Or, an application specific integrated circuit (ASIC) may replace one or more components.

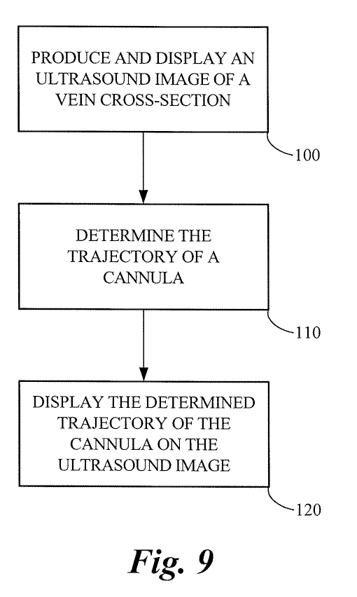

FIG. 9 is a flowchart of a process of displaying the trajectory of a cannula in accordance with an embodiment of the present invention. The process is illustrated as a set of operations shown as discrete blocks. The process may be implemented in any suitable hardware, software, firmware, or combination thereof. As such the process may be implemented in computer-executable instructions that can be transferred from one computer to a second computer via a communications medium. The order in which the operations are described is not to be necessarily construed as a limitation. First, at a block 100, an ultrasound image of a vein cross-section may be produced and/or displayed. Next, at a block 110, the trajectory of a cannula may be determined. Then, at a block 120, the determined trajectory of the cannula may be displayed on the ultrasound image.

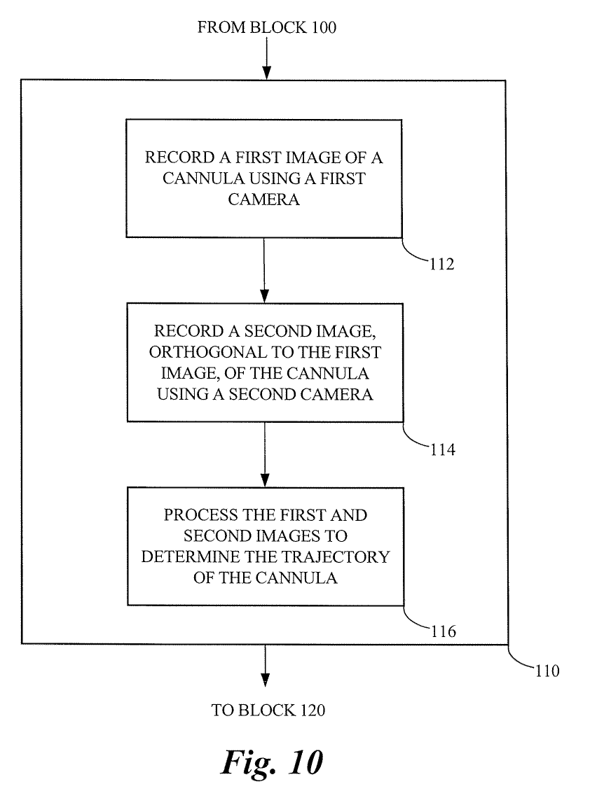

FIG. 10 is a flowchart of a process showing additional detail for the block 110 depicted in FIG. 9. The process is illustrated as a set of operations shown as discrete blocks. The process may be implemented in any suitable hardware, software, firmware, or combination thereof. As such the process may be implemented in computer-executable instructions that can be transferred from one computer to a second computer via a communications medium. The order in which the operations are described is not to be necessarily construed as a limitation. The block 110 includes a block 112 where a first image of a cannula may be recorded using a first camera. Next, at a block 114, a second image of the cannula orthogonal to the first image of the cannula may be recorded using a second camera. Then, at a block 116, the first and second images may be processed to determine the trajectory of the cannula.

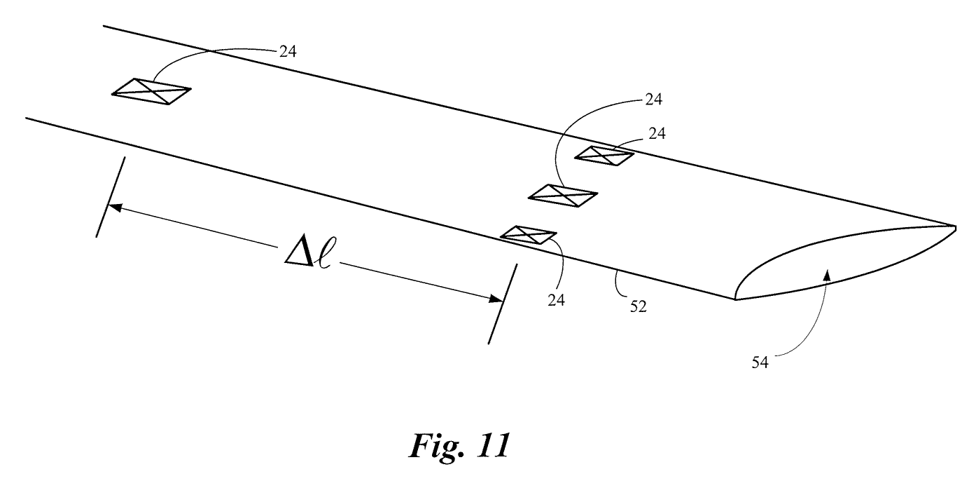

FIG. 11 schematically depicts an alternative embodiment of a needle having a distribution of reflectors located near the bevel of the needle. A needle shaft 52 includes a bevel 54 that may be pointed for penetration into the skin to reach the lumen of a blood vessel. The needle shaft 52 also includes a plurality of micro corner reflectors 24. The micro corner reflectors 24 may be cut into the needle shaft 52 at defined intervals Δl in symmetrical patterns about the circumference of the needle shaft 52. In an example, the micro corner reflectors 24 may be cut with a laser and serve to provide light reflective surfaces for monitoring the insertion and/or tracking of the trajectory of the bevel 54 into the blood vessel during the initial penetration stages of the needle 52 into the skin and/or tracking of the bevel 54 motion during guidance procedures.

While the preferred embodiment of the invention has been illustrated and described, as noted above, many changes can be made without departing from the spirit and scope of the invention. For example, a three-dimensional ultrasound system could be used rather than a 2D system. In addition, different numbers of cameras could be used along with image processing that determines the cannula 20 trajectory based on the number of cameras used. The two cameras 14, 18 could also be placed in a non-orthogonal relationship so long as the image processing was adjusted to properly determine the orientation and/or projected trajectory of the cannula 20. The radiation emitting from the light sources 26, 28 may be of a frequency and intensity that may be sufficiently penetrating in tissue to permit reflection of sub-dermal located reflectors 24 to the detector sensor 32. The sensor 32 may be suitably filtered to optimize detection of sub-dermal reflected radiation from the reflectors 24 so that sub-dermal trajectory tracking of the needles 22, 52 or cannulas 20 having one or more reflectors 24 may be achieved. Also, an embodiment of the invention could be used for needles and/or other devices such as trocars, stylets, or catheters which are to be inserted in the body of a patient. Additionally, an embodiment of the invention could be used in places other than arm veins. Regions of the patient's body other than an arm could be used and/or biological structures other than veins may be the focus of interest.

The embodiments of the invention in which an exclusive property or privilege is claimed are defined as follows:

1. A system for visualizing a medical object trajectory comprising:
a processing and display generating system;
an ultrasound probe for scanning tissue in signal communication with the processing and display generating system; and
at least one camera for capturing at least one image of a medical object in signal communication with the processing and display generating system,
wherein the processing and display generating system is configured to process signals received from the ultrasound probe, display an ultrasound image of the tissue, process signals received from the at least one camera to determine a trajectory of the medical object, and display a representation of the determined trajectory of the medical object on the ultrasound image.

2. The system of claim 1, wherein the at least one camera includes a first camera that takes images of the medical object in a first direction and a second camera that takes images of the medical object in a second direction.

3. The system of claim 2, wherein the first and second cameras are in a fixed position relative to the ultrasound probe.

4. The system of claim 3, wherein the second direction is orthogonal to the first direction.

5. The system of claim 2, wherein the medical object includes a cannula.

6. The system of claim 2, wherein the medical object includes a plurality of reflectors and wherein the processing and display generating system is configured to determine a trajectory of the medical object based on light reflected by the plurality of reflectors.

7. The system of claim 6, wherein the medical object includes a needle having a bevel, at least one of the reflectors is located near the bevel, and the processing and display generating system is configured to determine a trajectory of the bevel.

8. The system of claim 2, wherein the processing and display generating system is configured to display a cross-sectional image of a target vein located within the scanned tissue on the ultrasound image and wherein the processing and display generating system is configured to display a representation of the determined trajectory of the medical object on the ultrasound image.

9. The system of claim 8, wherein the representation of the determined trajectory includes cross-hairs.

10. The system of claim 2, further comprising an illumination source for illuminating the medical object during image capture.

11. The system of claim 10, wherein the illumination source includes infrared light emitting diodes.

12. The system of claim 2, wherein at least one of the first and second cameras are configured to capture images of a portion of the medical object when the portion is in a sub-dermal location.

13. A method for visualizing a medical object trajectory comprising:
scanning tissue using an ultrasound probe;
displaying an ultrasound image of the tissue;
capturing at least one image of a medical object using at least one camera;
processing the at least one image to determine a trajectory of the medical object; and
displaying a representation of the determined trajectory of the medical object on the ultrasound image.

14. The method of claim 13, wherein capturing at least one image includes capturing a first image of the medical object from a first camera in a first direction and capturing a second image of the medical object from a second camera in a second direction.

15. The method of claim 14, wherein the second direction is orthogonal to the first direction.

16. The method of claim 14, wherein the medical object includes a cannula.

17. The method of claim 14, wherein displaying an ultrasound image of the tissue includes displaying a cross-sectional image of the tissue scanned by a scanning plane of the ultrasound probe.

18. The method of claim 17, wherein processing the at least one image to determine a trajectory of the medical object includes determining a location where the medical object is projected to intersect the scanning plane, and wherein displaying a representation of the determined trajectory includes displaying cross-hairs on the cross-sectional ultrasound image at the projected intersection location.

19. A system for visualizing a medical object trajectory comprising:
ultrasound scanning means for scanning tissue;
image capture means for capturing at least one image of a medical object;
processing means for processing signals received from the ultrasound scanning means and for processing signals received from the image capture means to determine a trajectory of the medical object, the processing means in signal communication with the ultrasound scanning means and the image capture means; and
display generating means for displaying an image of the scanned tissue and a representation of the determined trajectory of the medical object on the image.

20. The system of 19, wherein the image capture means includes first image capture means for capturing images of the medical object in a first direction and second image capture means for capturing images of the medical object in a second direction.
